(19) United States
(12) Patent Application Publication
(10) Pub. No.: US 2022/0023325 A1
(43) Pub. Date: Jan. 27, 2022

(54) SPECIFIC TLR4 ANTAGONIST IN THE TREATMENT OF MULTIPLE MYELOMA

(71) Applicants: Etablissement Français du Sang, La Plaine Saint Denis (FR); Centre national de la recherche scientifique, Paris (FR); UNIVERSITE PAUL SABATIER TOULOUSE III, Toulouse Cedex 9 (FR); INSTITUT NATIONAL DE LA SANTE ET DE LA RECHERCHE MEDICALE (INSERM), Paris (FR)

(72) Inventors: Nicolas ESPAGNOLLE, Lapeyrouse Fossat (FR); Frédéric DESCHASEAUX, Toulouse (FR); Luc SENSEBE, Joue Les Tours (FR); Philippe BOURIN, Cugnaux (FR)

(21) Appl. No.: 16/472,754

(22) PCT Filed: Dec. 22, 2017
(86) PCT No.: PCT/EP2017/084457

§ 371 Date: Jun. 21, 2019

(30) Foreign Application Priority Data
Dec. 23, 2016 (FR) 1663344

(51) Int. Cl. A61K 31/7008; A61P 35/00

(52) U.S. Cl. CPC A61K 31/7008; A61P 35/00

(57) ABSTRACT

The present invention relates to a TLR4 (toll-like receptor 4) specific antagonist for use in the treatment of multiple myeloma in a subject suffering from multiple myeloma, and also to an antitumor pharmaceutical combination comprising (i) a TLR4-specific antagonist and (ii) a chemotherapy agent for simultaneous, separate or sequential use in the treatment of multiple myeloma.

The present invention relates to the treatment of multiple myeloma.

Multiple myeloma (MM) is a hematological cancer (or hematological malignancy) in which clonal plasma cells (myeloma cells) accumulate in the bone marrow.

Plasma cells are immune system cells derived from the bone marrow (BM) that produce antibodies to protect the body against external attacks (bacteria, viruses). During development, genetic abnormalities (deletion, chromosomal translocation) may occur, transforming healthy plasma cells into malignant plasma cells otherwise known as multiple myeloma cells. In the normal state, these plasmocytes circulate in the blood whereas in the pathology they return in the bone marrow where they cause damage on several levels.

Since many organs may be affected by multiple myeloma, the characteristics and signs of this disease are variable. The most frequent consequences include devastating bone lesions, high calcium levels, kidney failure, anemia, and altered immune abilities. Osteolytic lesions affect 80% of MM patients and remain a major cause of morbidity and mortality. The bone damage done to patients is the main problem in the long term. These bone lesions are caused by several factors: i) an important secretion of differentiation factors (IL-3, MIP-1α, MIP-1β, RANKL) allowing the transformation of monocytes into osteoclasts which are the cells responsible for bone degradation, and ii) inhibition by MM plasmocytes (by soluble factors such as DKK1 or TNFα) of the differentiation of mesenchymal stem cells into osteoblasts responsible for bone synthesis. Bone destruction also causes an increase in the level of calcium in the bloodstream, called hypercalcemia. In addition, because of the proliferation and congestion of myeloma cells in the bone marrow, the production of normal blood cells from the hematopoietic tissue is also impaired. Therefore, two consequences result, a decrease in the number of white blood cells (or leukocytes, responsible for immunity) which may increase the risk of infection, and a reduced production of red blood cells which may cause anemia. In addition, the excess of M proteins and light chain proteins constituting the antibodies, produced by myeloma cells thickens the blood resulting in thrombi and other circulatory problems. These proteins can also damage the kidneys and affect their proper functioning.

Multiple myeloma is the second most prevalent blood cancer after non-Hodgkin's lymphoma. It accounts for about 1% of all cancers, 10% of hematological malignancies and 2% of all deaths due to cancer.

MM is also characterized by a premyelomatous and asymptomatic phase designated MGUS for monoclonal gammopathy of undetermined significance. MGUS is the most common clonal plasma cell disease and transforms into MM with an incidence of 1% per year. Despite recent and numerous advances in pathology therapies, MM remains an incurable disease (no complete and sustainable remission) to date with a median survival of a few months to several years (5 years on average).

There is therefore a great need for new treatments for multiple myeloma.

The present invention aims to meet this need.

It is well established that BM and its constituents are required for the differentiation, maintenance, expansion and drug resistance of tumor plasmocyte clones. The microenvironment of BM is a complex network of heterogeneous cells that includes osteoclasts, lymphoid cells, endothelial cells, mesenchymal stromal cells (MSCs) and their offspring (osteoblasts and adipocytes). In MM, the balance between cellular and extracellular compartments within the BM is profoundly disrupted. In view of the literature, it is clearly established that MSCs play a leading role in the establishment and evolution of physiopathology. In fact, MSCs support the growth of myeloma plasma cells by producing high levels of interleukin-6 (IL-6), a major cytokine for the proliferation and survival of malignant plasma cells. These MSCs also play a role in the chemoresistance of MM plasma cells by conferring on them privileged niches or areas of protection against commonly used chemotherapeutic agents. The present inventors have previously shown that these medullary cells, present in the tumor microenvironment, presented abnormalities in patients with MM, such as overexpression of IL6 or GDF15 Corre et al. (2007) Leukemia 21: 1079-1088).

However, no medicament specifically targeting microenvironment, especially bone marrow MSCs in MM patients, exist to date.

The present inventors have surprisingly shown that the TLR4 protein (toll-like receptor 4) recognizing foreign (bacterial, viral) or endogenous (chaperone protein) motifs is overexpressed in MSCs of MM patients. Activation of this receptor in MSCs by its exogenous ligands (such as lipopolysaccharide, LPS) or endogenous ligands (such as Chaperone Heat shock protein 70, Hsp70) causes an increase in the expression of the CD54 adhesion protein (responsible for the interaction with MM plasmocytes) and interleukin 6 (responsible for the survival and proliferation of MM plasmocytes). After demonstrating that the activation of TLR4 was stronger in the MSCs of MM patients, the inventors used a specific TLR4 antagonist, the C34 compound, to study the impact on the behavior of MM MSCs. The inventors have thus shown that it is possible to alter the growth support capacity of the MSCs towards the myeloma cells with this inhibitor.

Thus, the inventors show here that it is possible to inhibit the growth of myeloma cells by acting only on the MSCs, more particularly on the fact that these MSCs overexpress TLR4, independently of the possible expression of this protein by the myeloma cells.

Moreover, the inventors have shown that a variability of expression of TLR4 by the MSCs was observable among patients suffering from MM.

Therefore, the use of TLR4-specific antagonists to alter the growth-supporting ability of MSCs to myeloma cells is particularly useful for treating subgroups of patients with higher TLR4 overexpression by MSCs. Moreover, this use targeting only MSCs and not malignant plasma cells, is particularly relevant for patients whose myeloma cells do not overexpress TLR4. Only one-third of the malignant plasma cells in patients actually express TLR4.

The present invention therefore relates to a TLR4-specific antagonist for use in the treatment of multiple myeloma in a subject suffering from this condition.

The present inventors have also surprisingly shown that the TLR4 antagonist acts synergistically with melphalan and lenalidomide, two chemotherapeutic agents commonly administered in MM patients and acting directly on MM plasma cells.

The present invention thus also relates to an antitumor pharmaceutical combination comprising (i) a TLR4-specific antagonist, typically targeting MSCs of the medullary microenvironment, and (ii) a chemotherapeutic agent, typically targeting MM plasma cells, for simultaneous, separate or sequential use in the treatment of multiple myeloma.

It also relates to a TLR4-specific antagonist for use in combination with a chemotherapeutic agent in the treatment of multiple myeloma.

It also relates to a chemotherapeutic agent for use in combination with a TLR4-specific antagonist in the treatment of multiple myeloma.

### DETAILED DESCRIPTION OF THE INVENTION

### Multiple Myeloma

By “multiple myeloma” is meant herein a cancer of the plasma cells. It may be asymptomatic or symptomatic.

Asymptomatic patients do not show disorders or symptoms associated with multiple myeloma in their tissues or organs. Tissue or organ disorders associated with MM include hypercalcemia, impaired renal function, anemia, and devastating bone damage. Asymptomatic myeloma is a premyelomatous phase that includes multiple smoldering myeloma (MGUS) and indolent multiple myeloma (smoldering myeloma or stage I multiple myeloma). MGUS for monoclonal gammopathy of undetermined significance (Ig<30 g/I and<10% plasma cells in BM) is the most common clonal plasma cell disease and transforms into MM with an incidence of 1% per year. Indolent multiple myeloma (smoldering myeloma or stage I multiple myeloma) corresponds to Ig≥30g/l and ≥10% plasma cells in the BM as well as a concentration of microglobulin β2 in the blood strictly below 3.5 mg/dl and an albumin concentration in the blood strictly greater than 3.5 g/dl. Indolent multiple myeloma is transformed into MM with an incidence of 10% per year.

Preferably, the subject treated in the context of the invention is a subject suffering from symptomatic multiple myeloma.

Patients with multiple myeloma may also be characterized by the status of their disease. The status of the disease may be determined on the basis of whether or not patients have ever received treatment, and if so, the effect of this treatment. Patients who have undergone therapy may be divided into several categories:
Responding disease: the myeloma responds to treatment and there is a decrease in protein M of at least 50%.
Stable disease: myeloma did not respond to treatment (i.e. the protein M decrease did not reach 50%) but did not worsen.
Progressive disease: myeloma is active and has worsened (i.e. increase in protein M and aggravation of disorders of tissues or organs). In most cases, a relapse and/or refractory illness may be considered a progressive disease.
Relapse of the disease: the myeloma that initially responded to the treatment then began to progress again.
Refractory disease: myeloma did not respond to initial therapy.

Preferably, the subject treated in the context of the present invention is a subject who has already received a treatment. More preferably, the subject is a subject suffering from stable or progressive myeloma, or a relapse of myeloma or refractory myeloma.

By “subject” is meant here a mammal, preferably a human. The present inventors have shown that a TLR4-specific antagonist can treat multiple myeloma by directly acting on mesenchymal stromal cells that overexpress TLR4, thereby decreasing their growth support capacity for myeloma cells.

The present invention is therefore particularly useful for treating a subgroup of subjects suffering from MM having an increased level of expression of the gene encoding the TLR4 protein at its MSC relative to the level of expression of the gene encoding TLR4 in the MSC of a healthy subject.

Thus, in a particular embodiment, the subject suffering from MM treated in the context of the present invention has an increased level of expression of the gene encoding the TLR4 protein at its MSC relative to the level of expression of the TLR4 coding gene in MSCs of a healthy subject.

By “TLR4”, “Toll Like Receptor 4” or “CD284” is meant here a membrane receptor of the family of TLR (and more broadly PRR—pattern recognition receptor) present on the majority of immune cells and some adipocytes. It is encoded by the TLR4 gene. TLR4 typically recognizes bacterial lipopolysaccharides (LPS) of gram-negative bacteria and endogenous ligands such as chaperone proteins (Hsp70).

By “healthy subject” is meant here a subject not suffering from multiple myeloma, symptomatic or asymptomatic, or from another pathology of bone marrow cells.

By “level of expression of the gene encoding the TLR4 protein” is meant here the level of transcribed mRNAs or proteins translated from the gene encoding the TLR4 protein. By “increased expression level of the gene encoding TLR4 protein” is meant here preferably a significantly increased level.

The level of expression of the mRNA encoding the TLR4 protein may be determined by any technique well known to those skilled in the art, for example by quantitative PCR or by means of DNA chips.

The level of expression of the TLR4 protein may be determined by any technique well known to those skilled in the art, for example by Western Blot, immunofluorescence or flow cytometry, using antibodies specifically directed against the TLR4 protein.

Since the inventors have shown that the TLR4 antagonist has a direct effect on MSCs, the subject treated in the context of the present invention does not necessarily have overexpression of the gene encoding the TLR4 protein at its tumor plasmocytes for that effect to be observed.

Thus, in a particular embodiment, the subject does not exhibit an increased level of expression of the gene encoding the TLR4 protein at its tumor plasmocytes.

### TLR4 Specific Antagonist

By “TLR4 specific antagonist” is meant here a compound inhibiting TLR4 activity, as defined above, without substantially inhibiting the activity of another TLR. Preferably, the TLR4-specific antagonist is not an inhibitor of TLR4 expression.

By “without substantially inhibiting the activity of another TLR” is meant herein that the activity of another TLR is inhibited by less than 20%, preferably less than 15%, less than 10%, less than 5%, more preferably less than 1%, in the presence of the antagonist.

In particular, preferably, the TLR4-specific antagonist does not substantially inhibit TLR1, TLR2, TLR3, TLR7, TLR8 or TLR9, in particular TLR3.

Techniques for determining the inhibition of TLR activity are well known to those skilled in the art and include, for example, the measurement of the expression of Interferon (IFN) regulatory factor 7 (IRF7), an intracellular regulator of production of TLR1, 2, 6, 7 and 9 mediated IFNα (as described in Wang et al (2016) Eur J.I. 46: 2409-2419).

TLR4-specific antagonists are well known to those skilled in the art and include the compound C34 of formula (I) below:

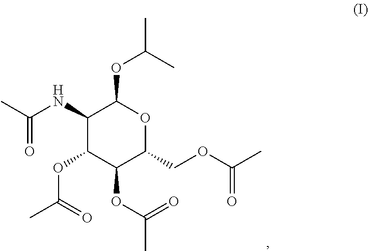 [SMILES: CC(=O)N[C@H]1[C@@H](OC(C)C)O[C@H](COC(C)=O)[C@@H](OC(C)=O)[C@@H]1OC(C)=O]

the following compounds described in US application US2016/281395: (2S-2-((4aR,6R,7R,8R,8aS)-7-acetamido-6 (2,3-bis(dodecyloxy))propoxy)-2,2-dimethylohexahydropyrano[3,2-d][1,3] dioxin-8-yloxy)propanoic acid, dodecyl3,4,6-tri-O-acetyl-2-(acetylamino)-2-deoxy-beta-D-glucopyranoside, butyl 2-(acetylamino)-2-deoxy-3,4-di-O-methyl-betal-D-glucopyranoside, isopropyl 3,4,6-tri-O-acetyl-2-(acetylamino)-2-deoxyhexopyranoside, cyclohexyl 3,4,6-tri-O-acetyl-2-(acetylamino)-2-deoxy-alpha-D-glucopyranoside, hexyl 3,4,6-tri-O-acetyl-2-(acetylamino)-2-deoxy-beta-D-glucopyranoside, N-[(2R,3R,4R,5S,6R)-2-[(1′S,2′R,6′R,8′R,9′S)-dispiro[cyclohexane-1,4′-[3,5,7,10,12]penta-oxatricyclo[7.3.0.0{2,6}]dodecane-11′1″-cyclohexane] -8′-ylmethoxy]-4,5-dihydroxy-6-(hydroxymethyl)oxan-3-yl]acetamide,(2R, 3S,4R,5R,6R)-5-acetamido-2-(acetoxymethyl))-6-(((3aR,5R,5aS,8aS,8bR)-2,2,7,7-tetramethyltetrahydro-3aH-bis[1,3] dioxolo[4,5-b:4′5′-d]pyran-5-yl)methoxy)tetrahydro-2H-pyran-3,4-diodi-diacetate, N-((2R,3R,4R,5S,6R)-4,5-dihydroxy-6-(hydroxymethyl)-2-(((3aR,5R,5aS,8aS,8bR)-2,2,7,7-tetramethyltetrahydro-3aH-bis[1,3]dioxolo[4,5-b:4′,5′-d]pyran-5-yl)methoxy)tetrahydro-2H-pyran-3-yl)acetamide, propyl3,4,6-tri-O-acetyl-2-(acetylamino)-2-deoxyhexopyranoside, 1,3,4,6-tetra-O-acetyl-2-deoxy-2-(palmitoylamino))hexopyranose, 6-O-[2-(acetylamino)-2-deoxy-beta-D-glucopyranosyl]-3-O-isopendyl-1,2-O-(1-methylethylidene)-alpha-D-xylo-hexofuranose, 6-O-[2-(acetylamino)-2-deoxy-beta-D-glucopyranosyl]-1,2-O-(1-methylethylidene)-3-O-propyl-alpha-D-xylo-hexofuranose1,2-O-(1-methylethylidene)-3-O-propyl-6-O-[3,4,6-tri-O-acetyl-2-(acetylamino)-2-deoxy-beta-D-glucopyranosyl]-alpha-D-xylo-hexofuranose, 1,2-O-(1-methylethylidene)-3-O-pentyl-6-O-[3,4,5-tri-O-acetyl-2-(acetylamino)-2-deoxy-beta-D-glucopyranosyl]-alpha-D-xylo-hexofuranose, octyl2-(acetylamino)-2-deoxy-beta-D-glycero-hexopyranoside, sec-butyl 2-(acetylamino)-2-deoxyhexopyranoside, (2S,4S,5R,6R)-5-acetamido-2-((2R,3S,4S,5R,6S)-3,5-dihydroxy-2-(hydroxymethyl)-64(2R,3S,4R,SS)-4,5,6-trihydroxy-2-(hydroxymethyl)tetrahydro-3H-pyran-3-yloxy), (2S,3S,4R,5R,6R)-3-((2S, 3R,5S,6R)-3-sodium-acetamido-5-hydroxy-6-(hydoymethyl)tetrahydro-2H-pyran-2-yloxy)-4,5,6-trihydroxytetrahydro-2H-pyran-2-carboxylate, 2-(acetylamino)-4-O-{2-(acetylamino)-4-O-[2-(acetylamino)-2-deoxy-beta-D-glucopyranosyl]-2-deoxy-beta-D-glucopyranosyl}-2-deoxy-D-glucopyranose, the sodium salt of 3-acetamido-4,5-dihydroxy-6-(hydroxymethyl)tetrahydro-2H-pyran-2-yldihydrogen phosphate, the compound sulfuric acid with (2R)-4-amino-N-{(1R,2S,3S,4R,5S)-5-amino-2-[(3-amino-3-deoxy-alpha-D-glucopyranosyl)oxy]-4-[(6-amino-[6-deoxy-alpha-D-glucopyranosyl)oxy]-3-hydroxycyclohexyl]-2-hydroxybutanamide (1:1), (4R)-4-((2S)-2-((2R)-2-((3R,4R,5S,6R)-3-acetamido-2,5-dihydroxy-6-(hydroxymethyl)tetrahydro-2H-pyran-4-yloxy)propanamido)propanamido)-5-amino-5-oxopentanoic acid, the sodium salt of uridine 5′-diphospho-N-acetylglucosamine, uridine disodium salt 5′-diphospho-N-acetylgalactosamine, 2-(acetylamino)-3-O-{4-O-[2-(acetylamino)-2-deoxy-3-0]alpha-D-xylo-hexopyranuronosyl-beta-D-ribo-hexopyranosyl]-beta-D-xylo-hexopyranuronosyl-2-deoxy-D-glucopyranose, 2-(acetylamino)-2-deoxy-3-O-(6,8-dideoxy-beta-L-glycero-octopyranosyl-7-ulose)-4-O-sulfo-L-erythro-hexopyranose, 2-(acetylamino)-2-deoxy-4-O-hexopyranosylhexopyranose, N-{(1S,2S, 3R)-1-[(beta-L-glycero-hexopyranosyloxy)methyl]-2,3-dihydroxyheptadecyl}hexacosanamide, dimethyl-5-(acetylamino)-3,5-dideoxy-D-erythro-non-2-ulopyranosidonate, methyl 2-(acetylamino)-2-deoxy-3-O-hexopyranosylhexopyranoside, 8-{[2-(acetylamino)-4-O-[2-(acetylamino)-2-deoxyhexopyranosyI]-2-deoxy-6-O-(6-deoxyhexopyranosyl)hexopyranosyl]oxyloctylacetate, octyl 2-(acetylamino)-2-deoxyhexopyranoside, 2-(acetylamino)-2-deoxy-4-O-(6-deoxyhexopyranosyl)-3-O-hexopyranosylhexopyranose, 2-(acetylamino)-2-deoxy-alpha-D-lyxo-hexopyranose, 2-(acetylamino)-2-deoxy-D-glucopyranose, allyl 3,4,6-tri-O-acetyl-2-(acetylamino)-2-deoxy-beta-D-lyxo-hexopyranoside, N-{(1S,2R,3E)-1-[(beta-L-ribo-hexopyranosyloxy)methyl]-2-hydroxy-3-heptadecenyl} octadecanamide, sodium ((3S, 6R)-5-acetamido-3,4,6-trihydroxytetrahydro-2H-pyran-2-yl)methyl phosphate, 2-((2R,SS)-3-acetamido-2,5-dihydroxy-6-(hydroxymethyl)tetrahydro-2H-pyran-4-yloxy)propanoic acid, allyl 2-(acetylamino)-2-deoxy-beta-D-glycero-hexopyranoside, 1,3,4,6-tetra-O-acetyl-2-(acetylamino)-2-deoxy-beta-D-glycero-hexopyranose, 2-(acetylamino)-2-deoxy-beta-D-glycero-hexopyranose, 4-O-[2-(acetylamino)-2-deoxyhexopyranosyl]-1,5-anhydro-2-deoxyhexitol, ethyl 2-(acetylamino)-2-deoxy-beta-D-glycero-hexopyranoside, ethyl 3,4,6-tri-O-acetyl-2-(acetylamino)-2-deoxy-beta-D-glycero-hexopyranoside, 5-acetamido-6-((1R,2R))-3-(3-(3-acetamido-5-hydroxy-6-(hydroxymethyl)-4-(3,4,5-trihydroxy-6-(hydroxymethyl)tetrahydro-2H-pyran-2-yloxy)tetrahydro-2H-pyran-2-yloxy)-6-(4,5-dihydroxy-6-((E)-3-hydroxy-2-stearamidooctadec-4-e, cyclohexanamine compound with 1,6-di-O-phosphono-beta-D-glycero-hexopyranose hydrate (4 : 1), 4-O-(3-O-{2-(acetylamino)-2-deoxy-4-O-(6-deoxyhexopyranosyl)-3-O-[2-O-(6-deoxyhexopyranosyl)} hexopyranosyl] hexopyranosyl} hexopyranosyl)hexopyranose, 3-O-(3-O-{2-(acetylamino)-2-deoxy-3-O-[2-O-(6-deoxyhexopyranosyl)hexopyranosyl] hexopyranosyl} hexopyranosyl)-D-arabinose, 2-(acetylamino)-2-deoxy-3-O-(6-deoxyhexopyranosyl)-4-O-hexopyranosylhexopyranose, nonyl 2-(acetylamino)-2-deoxy-beta-D-glycero-hexopyranoside octadecyl 2-(acetylamino)-2-deoxy-beta-D-glycero-hexopyranoside, 4-O-{6-O-[5-(acetylamino)-3,5-dideoxy-D-erythro-non-2-ulopyranonosyl] hexopyranosyl}hexopyranose, 2-deoxy-2-(propionylamino)-D-glucopyranose, the potassium salt and magnesium of cyclohexane-1,2,3,4,5,6-hexayl hexakis (dihydrogen phosphate), 1,3,4,6-tetra-O-acetyl-2-(acetylamino)-2-deoxy-beta-D-glucopyranose, 2-(acetylamino)-2-deo hydrate xy-D-galactopyranose, [(4R)-5-acetamido-3,4,6-triacetyloxy-oxan-2-yl] methyl acetate, [5-acetamido-3-acetyloxy-2-acetate] (acetyloxymethyl)-6-hexadecoxy-oxan-4-yl], (5-acetamido-3,4-diacetyloxy-6-pentoxy-oxan-2-yl)methyl acetate, (5-acetamido)acetate-3,4-diacetyloxy-6-methoxy-oxan-2-yl)methyl, N[-2-ethoxy-4,5-dihydroxy-6-(hydroxymethyl)oxan-3-yl] acetamide, acetate of [2,5-Diacetyloxy-6-(acetyloxymethyl)-3-(dodecanoylamino)oxan-4-yl], or N-[2-(dispiro [BLAH]ylmethoxy)-4,5-dihydroxy-6-(hydroxymethyl))oxan-3-yl] acetamide, (6R)-6-[[(2-chloro-4-fluorophenyl)amino]sulfonyl]-1-cyclohexene-1-carboxylic acid ethyl ester (TAK-242 or resatorvid ref: CLI-095, Invivogen), the freeze-dried VIPER peptide of sequence KYSFKLILAEYRRRRRRRRR (SEQ ID NO: 1)(VI PER sequence: KYSFKLILAEY (SEQ ID NO: 2))(Ref: NBP2-26244, Novus biologicals).

Alternatively, the TLR4-specific antagonist may be an antibody, including a conventional immunoglobulin, a single chain antibody, an Fab fragment, an Fv fragment, a single chain Fv (scFv) fragment or a nanobody, which antagonizes the activity of TLR4.

Examples of antagonist antibodies include neutralizing monoclonal antibody against human TLR4, clone W7C11 (mabg-ht1r4, Invivogen) and neutralizing rat polyclonal antibody against human TLR4 (pab-hstlr4, Invivogen).

Such an antibody may be produced by standard techniques. The ability of such an antibody to act as a TLR4 antagonist may be confirmed by the ability of the antibody to block an LPS-induced TLR4 activation index, such as an increase in CD54 expression (ICAM -1, Intercellular adhesion molecule -1) at the cell surface, increased production of secreted IL6 or phosphorylation of NFkB (Nuclear factor kappa B) key transcription factor of downstream signaling of TLR4.

Preferably, the TLR4-specific antagonist used in the context of the invention is the compound C34.

### Treatment of Multiple Myeloma

The present invention relates to a TLR4-specific antagonist, as defined above, for use in the treatment of multiple myeloma in a subject as defined above.

The present invention also relates to a method of treating multiple myeloma in a subject, comprising administering a therapeutically effective amount of a TLR4 specific antagonist as defined above in a subject in need thereof as defined above.

The present invention also relates to the use of a TLR4 specific antagonist, as defined above, for the manufacture of a medicament for the treatment of multiple myeloma in a subject as defined above.

The inventors have, in particular, shown that the TLR4 specific antagonist, by specifically targeting MSCs, makes it possible to inhibit the promoving effect of these cells on the proliferation of tumor plasmocytes.

Thus, in a particular embodiment, the TLR4 specific antagonist is used to inhibit the promoter effect of MSCs on the proliferation of tumor plasmocytes.

The treated multiple myeloma may be of any category as described above. Preferably, multiple myeloma is symptomatic multiple myeloma, more preferably progressive, relapsed and/or refractory multiple myeloma.

By “treatment” or “treating” is meant here the achievement, partially or substantially, of one or more of the following results: partially or totally reducing the extent of the disease, ameliorating a clinical symptom or indicator associated with the disease, delaying, inhibiting or preventing the progression of the disease, or partially or totally delaying, inhibiting or preventing the occurrence of a relapse of the disease.

By “therapeutically effective amount” is meant here an amount of active ingredient sufficient to destroy, modify, control or eliminate multiple myeloma. A “therapeutically effective amount” also refers to an amount of active ingredient needed to delay or minimize the extent of multiple myeloma. It also refers to the amount of active ingredient providing therapeutic benefit in the treatment or management of multiple myeloma. Finally, the term “therapeutically effective amount” means an amount of the active ingredient, alone or in combination with other therapies, which provides a therapeutic benefit in the treatment or management of multiple myeloma, including symptom improvement. associated with multiple myeloma.

In the context of the invention, the TLR4 specific antagonist may be used in combination with therapeutic support agents. By “therapeutic support agent” is meant here an agent to reduce the symptoms and complications of multiple myeloma. Examples of therapeutic support agents include bisphosphonates (acting on bone lesions), growth factors, antibiotics, diuretics and analgesics.

Examples of bisphosphonates include etidronate (Didronel), pamidronate (Aredia), alendronate (Fosamax), risedronate (Actonel), zoledronate (Zometa), and ibandronate (Boniva).

Examples of growth factors include G-CSF, GM-CSF, M-CSF, multi-colony stimulating factor, erythropoietin, thrombopoietin, oncostatin M and interleukins.

Examples of antibiotics include penicillins, cephalosporins and derivatives, oxolinic acid, amifloxacin, temafloxacin, nalidixic acid, piromidic acid, ciprofloxacin, cinoxacin, norfloxacin, perfloxacin, rosaxacin, ofloxacin, enoxacin, pipemidic acid, sulbactam, clavulinic acid, β-bromopenicillanic acid, β-chloropenicillanic acid, cephoxazole, sultampicillin, tazobactam, aztreonam, sulfazethine, isosulfazethine, norcardicines, chlortetracycline, oxytetracyline, tetracycline, demeclocycline, doxycycline, methacycline and minocycline.

Examples of diuretics include thiazide derivatives such as amiloride, chlorothiazide, hydrochlorothiazide, methylchlorothiazide and chlorothalidon.

Examples of analgesics include an opioid such as morphine, a COX-2 inhibitor, such as rofecoxib, valdecoxib and celecoxib, salicylates such as aspirin, magnesium cholinetrisalicylate, salsalate, and dirunisal. sodium salicylate, propionic acid derivatives such as fenoprofen, calcium, ibuprofen, ketoprofen, naproxen and naproxen sodium, indoleacetic acid derivatives such as indomethacin, sulfindac, etodalac and tolmetin, fenamates such as mefenamic acid and meclofenamate, benzothiazine derivatives or oxicams such as mobic or piroxicam, or pyrrolacetic acid such as ketorolac.

The TLR4 specific antagonist according to the invention and, optionally, the carrier therapeutic agent may be formulated in one or more separate pharmaceutical compositions as described below.

The TLR4 specific antagonist according to the invention may be administered by any suitable route of administration such as orally, sublingually, buccally, subcutaneously, transdermally, topically, intraperitoneally, intramuscularly, intravenously, subdermally or intranasally. Preferably, the TLR4 specific antagonist according to the invention is administered intravenously.

### Pharmaceutical Combination for Treating Multiple Myeloma

The inventors have also shown that the TLR4 specific antagonist, used in combination with a chemotherapeutic agent conventionally used for the treatment of multiple myeloma such as melphalan or lenalidomide, makes it possible to synergistically inhibit the proliferation of MM plasmocytes.

Thus, the present invention also relates to an antitumor pharmaceutical combination comprising (i) a TLR4 specific antagonist, as defined above, and (ii) a chemotherapeutic agent, for simultaneous, separate or sequential use in the treatment of multiple myeloma.

The present invention also relates to a method of treating multiple myeloma, comprising the simultaneous, separate or sequential administration of a therapeutically effective amount of (i) a TLR4 specific antagonist, as defined above, and ii) a chemotherapeutic agent, in a subject who needs it as defined above. The present invention also relates to the use of (i) a TLR4 specific antagonist, as defined above, and (ii) a chemotherapeutic agent for the manufacture of an antitumor pharmaceutical combination intended to be used simultaneously, separately or sequentially in the treatment of multiple myeloma, as defined above.

In the context of the invention, the term “combination”, “therapeutic combination” or “pharmaceutical combination” refers to either a fixed combination in the form of a dosage unit, or a “kit of parts” for combined administration where the TLR4 antagonist and the chemotherapeutic agent may be administered independently at the same time or separately within a time interval that allows the partners of the combination to show their synergistic effect.

The compounds of the combination may thus be formulated in one or more separate pharmaceutical compositions.

The present invention thus relates to an antitumor pharmaceutical composition comprising (i) a TLR4 specific antagonist as defined above, and (ii) a chemotherapeutic agent.

The present invention also relates to a kit comprising:
(i) a pharmaceutical composition comprising a TLR4 specific antagonist as defined above, and
(ii) a pharmaceutical composition comprising a chemotherapeutic agent.

The term “pharmaceutical composition” as defined herein means a mixture or solution comprising at least one therapeutic agent to be administered to a subject to prevent or treat a particular disease affecting the subject.

Typically, the compounds of the combination according to the invention may thus be combined with pharmaceutically acceptable excipients to form pharmaceutical compositions. The pharmaceutical compositions as defined above, therefore preferably further comprise pharmaceutically acceptable excipients.

By “pharmaceutically acceptable” is meant here compositions and molecular entities which do not produce side, allergic or otherwise undesired reactions when administered to a subject. A pharmaceutically acceptable excipient or vehicle is thus an encapsulant material, a diluent, a carrier, or any other non-toxic liquid, semi-solid or solid formulation aid.

In the pharmaceutical compositions used in the context of the present invention, for oral, sublingual, subcutaneous, intramuscular, intravenous, transdermal, local or rectal administration, the active ingredient, alone or in combination with another active ingredient, may be administered to the subject in a unit dosage form, as a mixture with conventional pharmaceutical carriers. Suitable unit dosage forms include oral forms such as tablets, gelled capsules, powders, granules and oral solutions or suspensions, sublingual or oral forms, aerosols, implants, for subcutaneous, transdermal, topical, intraperitoneal, intramuscular, intravenous, subdermal and intranasal routes.

The TLR4 specific antagonist and the chemotherapeutic agent may be administered simultaneously within the same composition or in different compositions. Alternatively, the TLR4 specific antagonist and chemotherapeutic agent may be sequentially administered, with the TLR4 specific antagonist being administered before or after the chemotherapeutic agent. The TLR4 specific antagonist and the chemotherapeutic agent may be administered by the same or different routes of administration.

Preferably, particularly when the pharmaceutical compositions are for parenteral administration, the pharmaceutical compositions contain pharmaceutically acceptable carriers for an injectable formulation. It may be, in particular, saline solutions, sterile, isotonic (monosodium or disodium phosphate, sodium chloride, potassium, calcium or magnesium, and mixtures of these salts)or dry compositions, in particular freeze-dried compositions which, after addition, according to the case of sterilized water or physiological saline solution, may be reconstituted into injectable solutions.

Dosage forms suitable for injectable use include sterile aqueous solutions or dispersions, formulations including sesame oil, peanut oil or aqueous propylene glycol, and sterile powders for extemporaneous preparation of solutions or dispersions of sterile injectables. Solutions comprising the compounds according to the invention in free base form or pharmaceutically acceptable salts may be suitably prepared in water with a surfactant such as hydroxypropylcellulose. The dispersions may also be prepared in glycerol, liquid polyethylene glycols, and mixtures thereof, and in oils. Under ordinary conditions of storage and use, these preparations preferably contain a preservative to prevent the growth of microorganisms.

The vehicle may also be a solvent or a dispersion medium containing, for example, water, ethanol, a polyol (for example glycerol, propylene glycol, liquid polyethylene glycol), mixtures that are adapted from those and vegetable oils. Acceptable fluidity may be maintained, for example, by using a coating, such as lecithin, by maintaining a required particle size in the case of dispersions and by using surfactants. The prevention of the action of microorganisms may be provided by various antibacterial and antifungal agents, such as parabens, chlorobutanol, phenol, sorbic acid, thimerosal, etc. In many cases, it is preferable to include isotonic agents, for example, sugars or sodium chloride. Prolonged absorption of injectable compositions may be provided by the use in absorption delaying agent compositions, such as aluminum monostearate or gelatin.

Sterile injectable solutions may be prepared by incorporating the active compound in a required amount of the appropriate solvent with several of the other ingredients mentioned above, if necessary, and then sterilizing by filtration. In general, the dispersions are prepared by incorporating the various sterilized active ingredients into a sterile vehicle which contains the basic dispersion medium and the other required ingredients among those mentioned above. In the case of sterile powders for the preparation of sterile injectable solutions, the preferred methods of preparation are vacuum drying and lyophilization techniques.

By “chemotherapeutic agent” is meant here an agent that is toxic to cancer cells. Examples of chemotherapeutic agents which may be used in the context of the invention include bortezomib (Velcade®, Millennium), melphalan, lenalidomide, predisone, vincristine, carmustine, cyclophosphamide, dexamathasone, thalidomide, pomalidomide, doxorubicin, cisplatin, etoposide and cytarabine. Preferably, the chemotherapeutic agent is a conventionally used agent depending on the stage or response to the treatment of MM such as melphalan, lenalidomide, bortezomib, thalidomide and pomalidomide.

In a particular embodiment, the chemotherapeutic agent is melphalan or lenalidomide, preferably lenalidomide.

The chemotherapeutic agent used in combination with the TLR4 specific antagonist according to the invention may be administered by any suitable route. Preferably, the chemotherapeutic agent, particularly melphalan or lenalinomide, is administered orally.

In a particularly preferred embodiment, the TLR4 specific antagonist according to the invention is administered intravenously and the chemotherapeutic agent, particularly melphalan or lenalinomide, is administered orally.

As indicated above, the TLR4 specific antagonist and the chemotherapeutic agent may be administered simultaneously or in a time-shifted manner, i.e. at different times and at equal or different time intervals for each of the members of the combination.

The ratio of the total amounts of the members (i) and (ii) of the combination may vary for example according to the needs of the patient.

The present invention also relates to a TLR4 specific antagonist, as defined above, for use in combination with a chemotherapeutic agent, as defined above, in the treatment of multiple myeloma, as defined above.

The present invention also relates to the use of a TLR4 specific antagonist, as defined above, for the manufacture of a medicament for use in combination with a chemotherapeutic agent, as defined above, in the treatment of multiple myeloma, as defined above.

The present invention also relates to a method of treating myeloma comprising administering, in a subject in need thereof, a therapeutically effective amount of a TLR4 specific antagonist as defined above, in combination with a chemotherapeutic agent as defined above.

The present invention also relates to a chemotherapeutic agent, as defined above, for use in combination with a TLR4 specific antagonist, as defined above, in the treatment of multiple myeloma, as defined above.

The present invention also relates to the use of a chemotherapeutic agent, as defined above, for the manufacture of a medicament for use in combination with a TLR4 specific antagonist, as defined above, in the treatment of multiple myeloma, as defined above.

The present invention also relates to a method of treating myeloma comprising administering, to a subject in need thereof, a therapeutically effective amount of a chemotherapeutic agent as defined above, in combination with a TLR4 specific antagonist, as defined above.

The present invention will be further illustrated by the figures and examples below.

### BRIEF DESCRIPTION OF THE FIGURES

FIG. 1: Level of expression of the mRNAs of the different variants (a), (b), (c) and (d) of TLR4 by the MSCs of healthy subjects, patients suffering from MGUS (pre-MM stage)or patients suffering from MM.

Statistics: *p≤1.05**p≤0.01, ***p≤0.001

FIG. 2: Expression of TLR4 protein on the surface of MSCs of healthy subjects and patients with MM. *p≤0.05

FIG. 3: Expression of different adhesion molecules involved in the interaction with MM plasma cells (CD49d, CD49e, CD54 and CD106) on the surface of MSCs of healthy subjects and patients with MM, stimulated or not with LPS. *p≤0.05**p≤0.01

FIG. 4: Hsp70 secretion level after one week of culture by MOLP-6 line (MM stroma-dependent plasma cells), MSCs of healthy donors or from patients suffering from MM. *p≤0.05

FIG. 5: Expression of CD54 by MSCs from healthy donors or from patients with MM, whether stimulated or not with Hsp70. p*≤0.05

FIG. 6: Secretion of IL6 by MSCs from healthy donors or from patients with MM, stimulated or not with LPS.

*p≤0.05**p≤0.01

FIG. 7: Secretion of IL6 by MSCs of healthy donors or from MM patients, whether stimulated or not with Hsp70.

*p≤0.05

FIG. 8: Number of myeloma cells (MOLP-6) after 7 days of coculture. MOLP-6 alone (without coculture), or in co-culture with stroma of MSCs from healthy donors or MM.

*p≤0.05

FIG. 9: Number of myeloma cells (MOLP-6)after culture of MOLP-6 alone untreated (NT) or treated with 1 μM or 10 μM of C34 (TLR4 antagonist), or in coculture with a stroma of MSCs from healthy or MM donors, untreated or treated with 1 μM or 10 μM C34.

**p≤0.01

FIG. 10: Expression of TLR4 protein on the surface of the MSCs of different patients with MM.

FIG. 11: Percentage inhibition of MM plasmocytes growth (MOLP6) in cocultures with MSCs from healthy donors (H; n=4) or untreated MM (MYE; n=5) treated (NT) with 1 μM of C34 (1 μM C34), with melphalan alone at 100 nM (Mel 100 nM), or with a combination of C34 at 1 μM and melphalan at 100 nM (Mel 100 nM+C34 1 μM). ns: non-significant; *p≤0.05; **p≤0.01

Statistical tests were performed with the Prism v5.02 software (t Student's test).

FIG. 12: Percentage inhibition of MM plasmocytes growth (MOLP6) in cocultures with MSCs from healthy donors (H; n=4) or untreated MM (MYE; n=5) treated (NT) with 1 μM of C34 (1 μM C34), with lenalidomide alone at 100 nM (Len 100 nM), or with a combination of

C34 at 1 μM and lenalidomide at 100 nM (Len 100 nM+C34 1 μM). ns: non-significant; *:p≤0.05; **:p≤0.01 Statistical tests were performed with the Prism v5.02 software (t Student's test).

### EXAMPLES

### Example 1

### Overexpression of TLR4 in MSCs of patients with MM

Following the transcriptomic analysis performed according to the method described in Corre et al. (2007) Leukemia 21: 1079-1088, the inventors have shown clear differences in TLR4 mRNA expression between healthy donor MSCs (healthy MSC) and patients with monoclonal gammopathies of undetermined significance (MGUS) (MGUS MSC), a multiple pre-myeloma condition, and secondly patients with multiple myeloma (MM MSC).

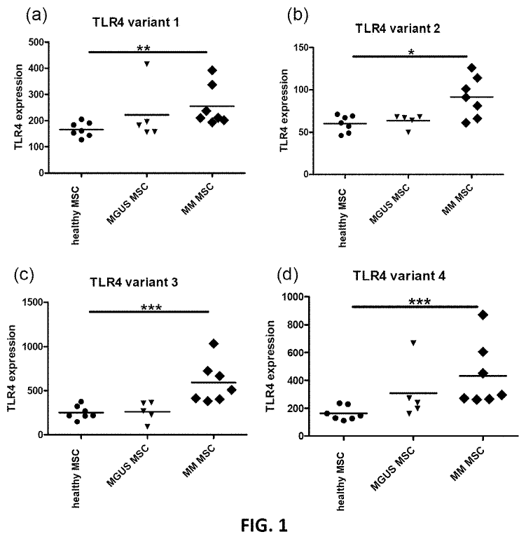

They therefore investigated the level of TLR4 in the MSCs and found that the mRNA level of 4 different variants of TLR4 was significantly higher in the MSCs of MM patients, compared to healthy donors and patients with MGUS (FIG. 1).

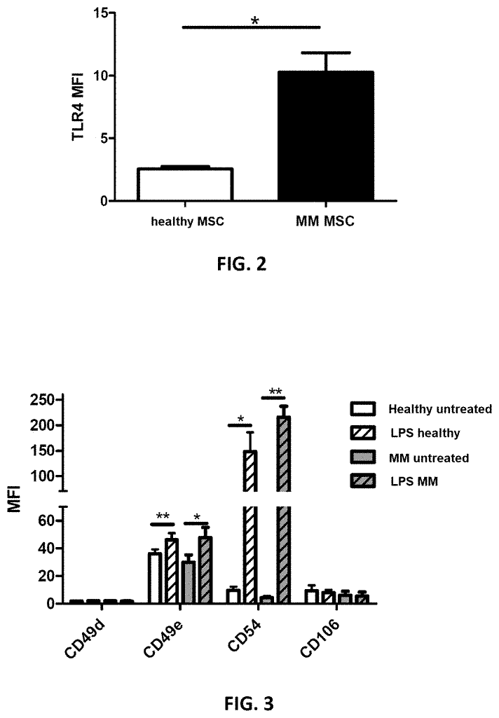

The inventors then analyzed the protein expression of TLR4 on the surface of MSCs of healthy subjects or MM patients after a 21-day MSC primoculture. They showed that TLR4 expression was higher on the surface of MM MSCs compared to healthy donor MSCs (MFI 10.3 v.s. 2.6, FIG. 2).

Thus, the TLR4 molecule is overexpressed both at the transcriptional level (mRNA) and at the protein level in the MM MSCs.

### Example 2

### Stimulation of TLR4 on MM MSCs Results in Higher Expression of CD54 and IL6 Secretion Compared to Healthy Donor MSCs

The inventors then studied the differential activation of TLR4 in the MSCs of healthy donors or MM patients. They analyzed the effect of stimulation by LPS, the TLR4 reference ligand widely described in the literature, on the expression of several adhesion molecules (CD49d/VLA-4, CD49e/VLA-5, CD54/ICAM-1 and CD106/VCAM-1) involved in the interaction between MSCs and malignant plasma cells. The level of expression (MFI: mean fluorescence intensity) of each molecule is shown in the histogram of FIG. 3.

The inventors observed that TLR4 stimulation by LPS had no effect on the expression of CD49d or CD106 but significantly increased the expression of CD49e and CD54 in healthy MSC and MM (FIG. 3). In fact, the stimulation of TLR4 by LPS strongly increases the expression of CD54 in healthy MSCs (CD54 MFI 9.6 vs 148.7) and on a larger scale in the MSCs of MM (CD54 MFI 4.3 vs 215.9).

A number of studies have evaluated the expression of adhesion molecules in MM-MSCs, but the results regarding the comparison of CD54 expression in healthy MSCs and MMs were contradictory. Here, the inventors have shown that, under basal conditions, the expression of CD54 was lower in the MM MSCs than in the healthy MSCs (MFI 4.3 vs. 9.6) but that after stimulation of TLR4 by LPS, CD54 expression was higher in MM MSCs than in healthy MSCs (MFI 216 vs. 149), suggesting a higher sensitivity of TLR4 or stronger downstream signaling of the molecule in MSCs of MM. Therefore, the regulation of CD54 expression in MSCs is disrupted in the context of MM.

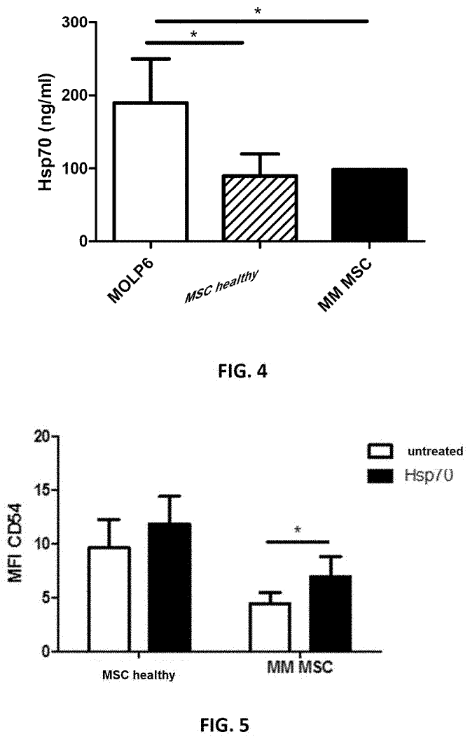

In order to further investigate the role of TLR4 in the context of MM and to move closer to pathophysiology, the inventors stimulated MSCs with Hsp70, an endogenous ligand of TLR4 actively released in the MM microenvironment and secreted by the cell line of MM MOLP-6 (up to 200 ng/ml) (FIG. 4). In addition, this endogenous ligand has been described in the literature to have a chemoprotective role for MM malignant plasma cells (Nimmanapalli et al (2008) Br. Haematol 142: 551-561). Interestingly, stimulation with Hsp70 significantly increased CD54 expression only in the MM MSCs (FIG. 5) confirming their greater sensitivity to TLR4. Indeed Hsp-70 is a monomeric molecule and therefore induces a less important stimulation than LPS (multimeric). Nevertheless, once again, the MM MSCs respond better than the healthy MSCs.

The inventors also evaluated the effect of TLR4 stimulation on the soluble IGF-1 and IL6 factors involved in the survival and proliferation of myeloma cells.

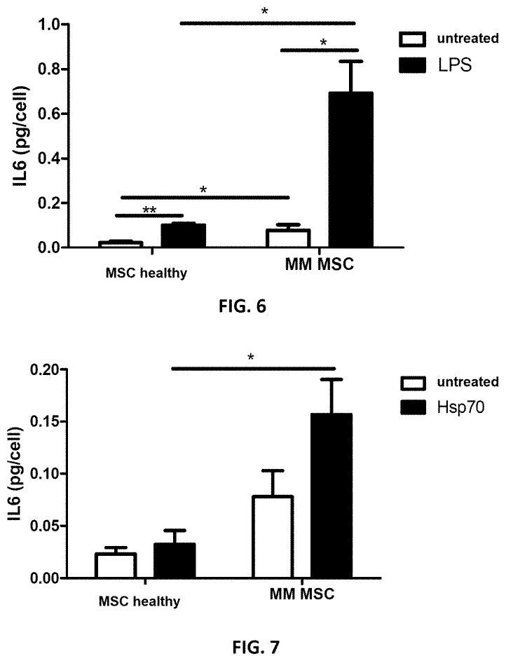

TLR4 stimulation by LPS has no effect on IGF-1 secretion in healthy and MMMCs but induces secretion of IL6 (major cytokine of MM cell survival and proliferation) in healthy MSCs and to a greater extent in the MSCs of MM (healthy MSCs 0.02 vs 0.1 pg/cell, MSCs of MM 0.08 vs 0.7 pg/cell) (FIG. 6).

As with the expression of CD54, Hsp70 increases IL6 secretion more strongly and specifically in MM MSCs (FIG. 7).

Therefore, IL6 secretion is higher in the MMSCs in which TLR4 is stimulated, confirming the hypersensitivity of TLR4 in these cells.

Thus, following the stimulation of TLR4, healthy MSCs and to a greater extent MM MSCs upregulate the soluble and adherent factors involved in supporting myeloma cell growth. In the bone marrow of MM patients, therefore, the MSCs could be chronically activated by endogenous ligands of TLR4 such as Hsp70. The latter is released on the one hand by cells damaged in bone lesions (apoptotic or injured cells) but also actively by MM plasma cells (as seen in FIG. 4) inducing upregulation of IL6 by the microenvironment MSCs. which therefore promotes the proliferation of myeloma cells.

### Example 3

### TLR4 Plays a Pivotal Role in the Support of MM MSCs for the Growth of MM Plasmocytes

To evaluate the role of TLR4 in the MSC/myeloma cell interaction, the inventors used a specific TLR4 antagonist, the C34 compound, marketed by Tocris, and a stroma-dependent myeloma cell line, the MOLP-6 line. Some studies have used stroma-independent myeloma cell lines such as MM1S or RPM18226. In this study, in order to approach the pathology and mimic real interactions between MSC and plasma cells, the inventors used a stroma-dependent cell line for its proliferation. Indeed, during MM (in the establishment and maintenance phases of the disease), myeloma cells strongly adhere to the MSCs of the stroma, which gives chemoresistance and survival signals favoring the evolution of MM.

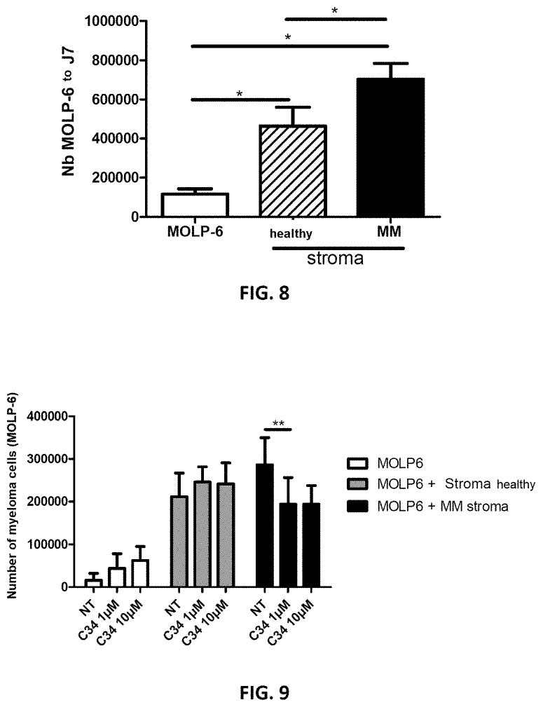

The inventors first of all confirmed that the MOLP-6 cells used for the study were indeed stroma-dependent (FIG. 8). In fact, MOLP-6 cells alone cannot proliferate but enter the cycle when co-cultured with a stroma. It could be noted that the stroma of MM supported the growth of MOLP-6 more than the healthy stroma as previously described in the literature (number of MOLP-6 cells at day 7:7.04×10⁵ vs 4.64×10⁵).

Prior to using the TLR4 antagonist on the stroma, the inventors verified that C34 did not directly affect MOLP-6 cells (FIG. 9). Then they treated the healthy MSCs and MM with the C34 before and in co-culture with the MOLP-6. With the healthy stroma, the C34 antagonist (at 1 and 10 μM) has no effect on the growth support of MOLP-6, whereas with the stroma of MM, the TLR4 antagonist affects growth of MOLP-6 cells at the two doses used (33% decrease, FIG. 9).

Therefore, the MSCs of MM are more sensitive to the TLR4 antagonist than the healthy MSCs in terms of supporting the proliferation of MOLP-6 cells. The originality of the results is also in the specificity of the effect of the product on the pathological MSCs.

Therefore, these results confirm that a TLR4 antagonist may be used to treat multiple myeloma by acting preferentially on pathological MSCs, affecting their growth support capacity of malignant plasma cells.

### Example 4

### Study of the Expression of TLR4 on MSCs of Patients with MM

The inventors studied TLR4 expression levels in the MSCs of different MM patients (Mye161, 165, 110, 168 and 106 patients) by flow cytometry.

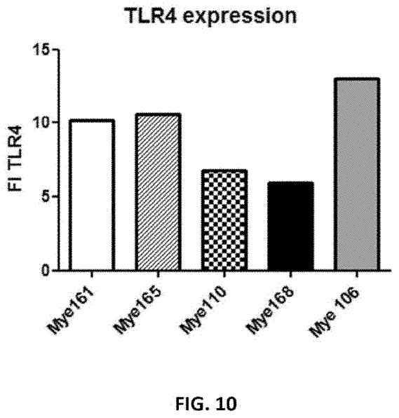

They showed that the expression of TLR4 was homogeneous within the same patient and that there was therefore no subpopulation of MSCs expressing differently TLR4 at the patient level. In contrast, they were able to observe fluctuations in TLR4 expression intensity between MM patients (FIG. 10).

This study therefore shows that it is possible to categorize MM patients according to the strong or weak expression of TLR4 by MSCs.

### Example 5

### Study of a Dual Therapy Combining a Chemotherapeutic Agent and a TLR4 Antagonist on a co-culture of MSCs/MM Plasma Cells

The inventors co-cultured MSCs from 4 healthy donors (H) or 5 patients suffering from MM (MYE) as well as stroma-dependent MOLP-6 plasma cells:
without treatment (NT)
in the presence of C34 alone at 1 μM (C34 1 μM),
in the presence of melphalan alone at 100 nM (Mel 100 nM),
in the presence of lenalidomide alone at 100 nM (Len 100 nM),
in the presence of a combination of C34 at 1 μM and melphalan at 100 nM (Mel 100 nM+C34 1 μM), or
in the presence of a combination of C34 at 1 μM and lenalidomide at 100 nM (Len 100 nM+C34 1 μM).

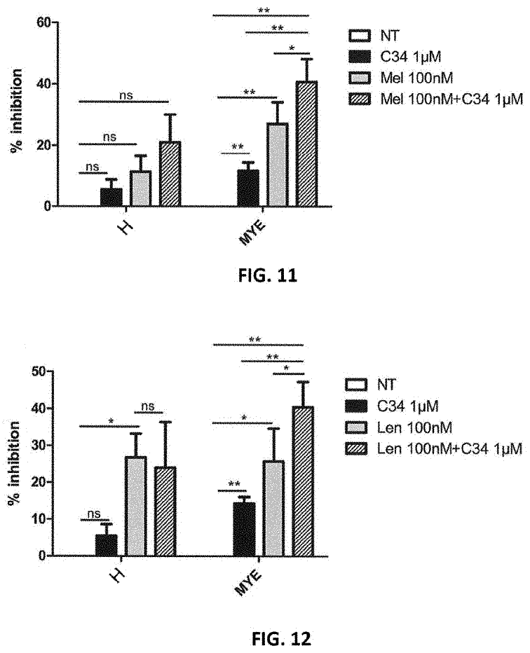

After 7 days of culture, they calculated the percent inhibition of plasmocyte growth (FIGS. 11 and 12).

The statistics were evaluated by the t Student test between the different conditions. They thus observed a synergistic effect of C34 on the inhibition of the proliferation of plasmocytes of MM cocultivated with MM MSC in combination with low doses of melphalan (100nM) (FIG. 11). The same results were noted with lenalidomide at 100nM (FIG. 12). Interestingly, no significant synergistic effect of C34 could be observed in healthy MSC/MOLP-6 cocultures in the presence of chemotherapeutic agents.

Interestingly, and in parallel with the results with C34 alone (FIG. 9), this synergistic effect is only observed on plasma cells co-cultured with MM MSCs. In addition, the effect of C34 is sustained in the presence of low concentration (100 nM) chemotherapeutic agents. Thus, another advantage of dual therapy is the fact that thanks to the combined action of C34, it is possible to reduce the doses of chemotherapeutic agents (melphalan and Lenalidomide at 100nM) potentially resulting in fewer side effects in patients.

What is claimed is:

1. A method of treating multiple myeloma in a subject, comprising administering a therapeutically effective amount of a TLR4-specific antagonist (toll-like receptor 4) to a subject in need thereof.

2. The method according to claim 1, wherein the subject has an increased level of expression of the gene encoding TLR4 protein at its mesenchymal stromal cells relative to the expression level of the TLR4 encoding gene in the mesenchymal stromal cells of a healthy subject.

3. The method according to claim, wherein the subject does not exhibit an increased level of expression of the gene encoding TLR4 protein at its tumor plasmocytes.

4. The method according to claims 1 for inhibiting the mesenchymal stromal cell promoting effect on proliferation of tumor plasmocytes.

5. The method according to claims 1 4, said TLR4-specific antagonist being the compound C34 of formula (I) below:

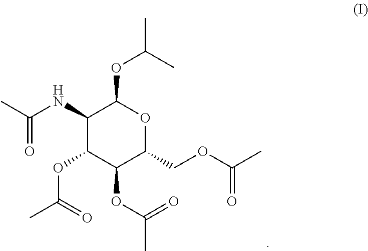 [SMILES: CC(=O)N[C@H]1[C@@H](OC(C)C)O[C@H](COC(C)=O)[C@@H](OC(C)=O)[C@@H]1OC(C)=O]

6. A method of treating multiple myeloma, comprising the simultaneous, separate or sequential administration of a therapeutically effective amount of (i) a TLR4-specific antagonist and (ii) at least one chemotherapeutic agent, in a subject in need thereof.

7. The method according to claim 6, wherein the TLR4-specific antagonist is the compound C34 of the following formula (I):

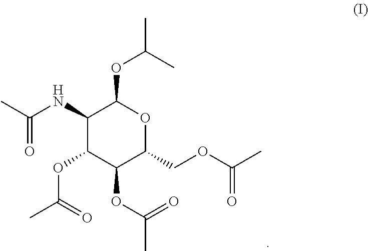 [SMILES: CC(=O)N[C@H]1[C@@H](OC(C)C)O[C@H](COC(C)=O)[C@@H](OC(C)=O)[C@@H]1OC(C)=O]

8. A method of treating multiple myeloma comprising administering, in a subject in need thereof, a therapeutically effective amount of a TLR4-specific antagonist in combination with at least one chemotherapeutic agent.

9. A method of treating multiple myeloma comprising administering, in a subject in need thereof, a therapeutically effective amount of a chemotherapeutic agent in combination with a TLR4-specific antagonist.


SEQUENCE LISTING (2 sequences)

SEQ ID NO: 1 - protein, 20 aa, Artificial Sequence
  freeze-dried VIPER peptide
KYSFKLILAEYRRRRRRRRR

SEQ ID NO: 2 - protein, 11 aa, Artificial Sequence
  VIPER peptide
KYSFKLILAEY